(19) United States
(12) Patent Application Publication
(10) Pub. No.: US 2017/0100485 A1
(43) Pub. Date: Apr. 13, 2017

(54) METAL-POLYSACCHARIDE CONJUGATES: METHODS FOR CANCER THERAPY

(71) Applicants: Taiwan Hopax Chemicals Mfg. Co., Ltd., Kaohsiung City (TW); BOARD OF REGENTS, THE UNIVERSITY OF TEXAS SYSTEM, AUSTIN, TX (US)

(72) Inventors: David J. Yang, Sugar Land, TX (US); Dong-Fang Yu, Houston, TX (US); I-Chien Wei, Kaohsiung City (TW)

(73) Assignees: Taiwan Hopax Chemicals Mfg. Co., Ltd., Kaohsiung City (TW); BOARD OF REGENTS, THE UNIVERSITY OF TEXAS SYSTEM, AUSTIN, TX (US)

(21) Appl. No.: 15/346,736

(22) Filed: Nov. 9, 2016

Related U.S. Application Data
(62) Division of application No. 12/009,421, filed on Jan. 18, 2008, now abandoned.
(60) Provisional application No. 60/933,034, filed on Jun. 4, 2007.

(51) Int. Cl. A61K 31/715; A61K 31/282; A61K 33/24

(52) U.S. Cl. CPC A61K 47/4823; A61K 31/282; A61K 33/24

(57) ABSTRACT

The current disclosure, in one embodiment, includes a polysaccharide conjugate. This conjugate has a polysaccharide and at least one monomeric amino acid having an O-group covalently bound to the polysaccharide. The conjugate also has at least one metal conjugated by the O-group of the amino acid. According to another embodiment, the disclosure provides a method of synthesizing a polysaccharide conjugate by covalently bonding a monomeric amino acid having an O-group to a polysaccharide and by conjugating a metal to the O-group to form a polysaccharide conjugate. According to a third embodiment, the disclosure relates to a method of killing a cancer cell by administering to the cell an effective amount of a polysaccharide conjugate. This conjugate has a polysaccharide and at least one monomeric amino acid having an O-group covalently bound to the polysaccharide. The conjugate also has at least one metal conjugated by the O-group of the amino acid.

### CROSS-REFERENCE TO RELATED APPLICATION

This application is a divisional application of and claims the benefit of U.S. Application Ser. No. 12/009,421, filed on Jan. 18, 2008, which claims the benefit of U.S. Provisional Application No. 60/933,034, filed on Jun. 4, 2007. The entirety of each of the above-mentioned patent applications is hereby incorporated by reference herein and made a part of this specification.

### TECHNICAL FIELD

Embodiments of the present invention relate to conjugates of metal and polysaccharides via monomeric amino acids. These polysaccharide conjugates may be used to induce cancer cell death and in cancer therapy.

### TECHNICAL BACKGROUND

Angiogenesis processes are involved in the tumor vasculature density and permeability. An increased understanding of these processes as well as cell cycle regulation and cell signaling agents has opened a new era in the treatment of various tumors. Despite the outstanding advances made in the field of angiogenesis, some significant limitations still remain in the treatment of cancer, tumors and other diseases having an angiogenic component via drug agents. One of the most significant limitations at this time relates to the delivery of cytotoxic drugs instead of cytostatic drugs in vivo.

The effectiveness of platinum conjugates against tumor activity has been demonstrated. For instance, cisplatin, a widely used anticancer drug, has been used as alone or in combination with other agents to treat breast and ovarian cancers. Cisplatin, also known as cis-diamminedichloroplatinum (II) (CDDP), is a simple molecule with Pt conjugated to NH₃ molecules. Cisplatin causes cell arrest at S-phase and that leads to mitotic arrest of proliferating cells. Cisplatin also decreases expression of vascular endothelial growth factor (VEGF) during chemotherapy, thus limiting angiogenesis. Cisplatin is effective in the treatment of majority solid tumors. However, clinical applications using cisplatin are limited due to significant nephrotoxicity, myelosuppression, drug resistance, gastrointestinal toxicity, neurotoxicity and other side effects (e.g. vomiting, granulocytopenia and body weight loss). In addition, cisplatin is formulated in bulky vehicles with poor water solubility, which impairs its therapeutic efficacy. Chemical modifications of various platinum conjugates have been made to increase its hydrophilicity, reduce its side effects and improve its therapeutic efficacy, however, these conjugates still present serious drawbacks.

### SUMMARY

The current invention, in one embodiment, includes a polysaccharide conjugate. This conjugate has a polysaccharide and at least one monomeric amino acid having an O-group covalently bound to the polysaccharide. The conjugate also has at least one metal conjugated by the O-group of the amino acid.

According to another embodiment, the invention provides a method of synthesizing a polysaccharide conjugate by covalently bonding a monomeric amino acid having an O-group to a polysacchride and by conjugating a metal to the O-group to form a polysaccharide conjugate.

According to a third embodiment, the invention relates to a method of killing a cancer cell by administering to the cell an effective amount of a polysaccharide conjugate. This conjugate has a polysaccharide and at least one monomeric amino acid having an O-group covalently bound to the polysaccharide. The conjugate also has at least one metal conjugated by the O-group of the amino acid.

### BRIEF DESCRIPTION OF THE DRAWINGS

The following figures form part of the present specification and are included to further demonstrate certain aspects of the present invention. The invention may be better understood by reference to one or more of these drawings in combination with the description of embodiments presented herein. The patent or application file contains at least one drawing executed in color. Copies of this patent or patent application publication with color drawing(s) will be provided by the Office upon request and payment of the necessary fee.

FIG. 1A, FIG. 1B, and FIG. 1C illustrate three types of metal-polysaccharide conjugates according to embodiments of the present invention. “AA” designates an amino acid. “M” designates a metal. In FIG. 1A, only one or a few amino acid groups and conjugated metal are present. In FIG. 1B, an intermediate number of amino acid groups and conjugated metal are present. In FIG. 1C the maximum or nearly the maximum possible amino acid groups and conjugated metal are present.

FIG. 2 shows one method (Method A) of synthesis of a platinum analogue (II) and (IV)-polysaccharide conjugate, according to an embodiment of the present invention.

FIG. 3 shows another method (Method B) of synthesis of a polysaccharide-platinum analogue (II) and (IV) conjugate, according to an embodiment of the present invention.

FIG. 4 shows the effect of a platinum-polysaccharide conjugate, according to an embodiment of the present invention, on inhibition of platinum-resistant ovarian cancer cells (2008 c13) at 48 hours (A) and 72 hours (B).

FIG. 5A and FIG. 5B show the effect of a platinum-polysaccharide conjugate, according to an embodiment of the present invention, on inhibition of platinum-sensitive ovarian cancer cells (2008) at 48 h (A) and 72 h (B).

FIG. 6A and FIG. 6B show the results of flow cytometry showing the apoptotic effects of cisplatin (CDDP) (A) and a platinum-polysaccharide conjugate (PC), according to an embodiment of the present invention, (B) on a platinum-resistant ovarian cancer cell line, 2008-c13, at 48 hours.

FIG. 7 shows the percentages of apoptotic cells detected by flow cytometry in the platinum-resistant ovarian cancer cell line 2008-c13 treated with a platinum-polysaccharide conjugate (PC), according to an embodiment of the present invention, or cisplatin (CDDP) at various concentrations for 48 hours (A) and 72 hours (B).

FIG. 8 shows the percentage of apoptotic cells detected by TUNEL assay in the platinum-resistant ovarian cancer cell line 2008-c13 treated with a platinum-polysaccharide conjugate (PC), according to an embodiment of the present invention, or cisplatin (CDDP) at various concentrations for 48 hours.

FIG. 9A and FIG. 9B show the in vivo effects of a platinum-polysaccharide conjugate, according to an embodiment of the present invention, against breast tumor growth at 24 hours (A) and 94 his (B) (single dose, Pt 10 mg/kg). The tumors designated DY were taken from an animal administered only chondriotin. The tumors designated DP-A-P were taken from an animal administered the platinum-polysaccharide conjugate. In FIG. 9A, the tumor on the left measured (2.08 cm×2.58 cm×1.96 cm)/2 for a volume of 5.2591 cm³. The tumor on the right measured (2.20 cm×2.37 cm×2.02 cm)/2 for a volume of 5.2661 cm³. In FIG. 9B, the tumor on the left measured (2.99 cm×3.29 cm×2.92 cm)/2 for a volume of 14.3622 cm³. The tumor on the right measured (1.11 cm×1.84 cm×0.86 cm)/2 for a volume of 0.8782 cm.

FIG. 10A, FIG. 10B, FIG. 10C, and FIG. 10D show H & E staining of tumors to show necrosis at 24 and 94 his post-administration of a platinum-polysaccharide conjugate, according to an embodiment of the present invention, or chondroitin alone. FIG. 10A shows a mammary tumor (13762) at 24 hrs administered chondroitin. FIG. 10B shows a mammary tumor (13762) at 24 his administered a platinum-polysaccharide conjugate. FIG. 10C shows a mammary tumor (13762) at 94 hrs administered chondroitin. FIG. 10D shows a mammary tumor (13762) at 94 hrs administered a platinum-polysaccharide conjugate.

FIG. 11 shows a Western blot of PARP protein from 2008-c13 cells treated with either platinum-polysaccharide conjugate (PC) or cisplatin (CDDP).

FIG. 12 shows the results of flow cytometric analysis of the cell cycle of 2008-c13 cells platinum-polysaccharide conjugate (PC) or cisplatin (CDDP) after 48 hours.

FIG. 13A and FIG. 13B show a Northern blot for p21 transcript (FIG. 13A) and a Western blot for expressed p21 (FIG. 13B) in 2008-c13 cells treated with low doses of platinum-polysaccharide conjugate (PC) or cisplatin (CDDP).

FIG. 14A shows Flow cytometric analysis of the dose-dependent increase of the sub-G₁ fraction after 48 hours-exposure to cisplatin (CDDP) or conjugate (PC or DDAP). At the same doses, PC induced substantially more sub-GI cells than did CDDP in platinum-resistant 2008.C13 cells. FIG. 14B shows the percentage of the sub-G₁ fraction in 2008.C13 cells after 48 hours-exposure to CDDP or PC (DDAP).

FIG. 15A and FIG. 15B show a TUNEL assay of apoptosis induced by cis-diamminedichloroplatinum(II) (CDDP) and diammine dicarboxylic acid platinum (PC or DDAP) after 48 hours of drug exposure. In FIG. 15A, the apoptotic morphology is indicated by brown particles. In FIG. 15B, the percentage of cells with apoptotic morphology. Columns, mean of three independent experiments; bars, SE.

FIG. 16 shows a Western blot analysis of cleaved caspase-3 and specific poly (ADP-ribose) polymerase (PARP) cleavage in 2008.C13 cells treated with cis-diamminedichloroplatinum(II) (CDDP) or diammine dicarboxylic acid platinum (PC or DDAP). GAPDH, glyceraldehyde-3-phosphate dehydrogenase.

FIG. 17A shows the cell-cycle distribution after treatment with cis-diamminedichloroplatinum(II) (CDDP) or diammine dicarboxylic acid platinum (PC or DDAP) for 48 hours in the 2008.C13 cell line. G₁, G₂, M, and S indicate cell phases. FIG. 17B shows a Western blot analysis of p21 and cyclin A expression in 2008.C13 cells after exposure to cis-diamminedichloroplatinum(II) (CDDP) or diammine dicarboxylic acid platinum (PC or DDAP) for 48 hours. GAPDH, glyceraldehyde-3-phosphate dehydrogenase.

### DETAILED DESCRIPTION

The present invention, in certain embodiments, includes metal-polysaccharides conjugates, methods for their synthesis, and uses thereof, including inducing cancer cell death and treatment of cancer. In particular, the conjugate may include a polysaccharide with at least one monomeric amino acid attached. This amino acid may then conjugate the metal. In selected embodiments, it may conjugate the metal via an O-group rather than a N-group. In some embodiments, the metal may be a transition metal. In many embodiments, there may be multiple monomeric amino acids attached, which allows for conjugation of multiple metal groups. The conjugates may be of any size, but in certain embodiments, the conjugate may be designed so that each molecule is at least 10,000 Daltons, for example between 10,000 and 50,000 Daltons, to limit excretion through the kidneys. In a particular embodiment, the polysaccharide conjugate may have a molecular weight of between about 20,000 Daltons to about 50,000 Daltons, more particularly it may be between about 26,000 to about 30,000 Daltons.

The polysaccharide selected may be any polysaccharide, but polysaccharides involved in vascular uptake may be particularly useful. In particular, adhesive molecules, such as collagen, chondroitin, hyauraniate, chitosan, and chitin may be well suited for use as the polysaccharide. In one particular embodiment, it may he chondroitin A. Although the present invention is not limited to a particular mode of action, such polysaccharides may facilitate uptake through the vasculature and delivery to cancer cells. This may be particularly true in areas undergoing angiogenesis, such as most tumors. The end product molecular weight range of 20,000-50,000 Daltons will help achieve vascular-based therapy.

The amino acid may be attached to the polysaccharide in any stable manner, but in many embodiments it will be covalently bonded to the polysaccharide. The amino acid may be in monomeric form, such that individual monomers are attached separately to the polysaccharide. The amino acid may have a O-group available for conjugation of the metal, in particular, it may have two O-groups available. The metal may be conjugated by a single monomeric amino acid, or via two or more monomeric amino acids. Example amino acid monomers that may be used alone or in combination include: glutamic acid, aspartic acid, glutamic acid combined with alanine, glutamic acid combined with asparagine, glutamic acid combined with glutamine, glutamic acid combined with glycine, and aspartic acid combined with glycine. Due to bond distance between two carboxylic acid and better tumor uptake specificity, aspartic acid is preferred. The amino acids may be in L-form, or D-form, or a racemic mixture of L- and D-forms. Amino acid in L-form is preferred for optimal tumor uptake. Aspartic acid may be selected because a single aspartic acid monomer is able to conjugate a metal on its own. Additionally, aspartic acid is not produced by mammalian cells, but is a necessary nutrient, making it likely to be taken up by rapidly growing tumor cells.

The amino acid may comprise between about 10% to about 50%, by weight of the polysaccharide conjugate.

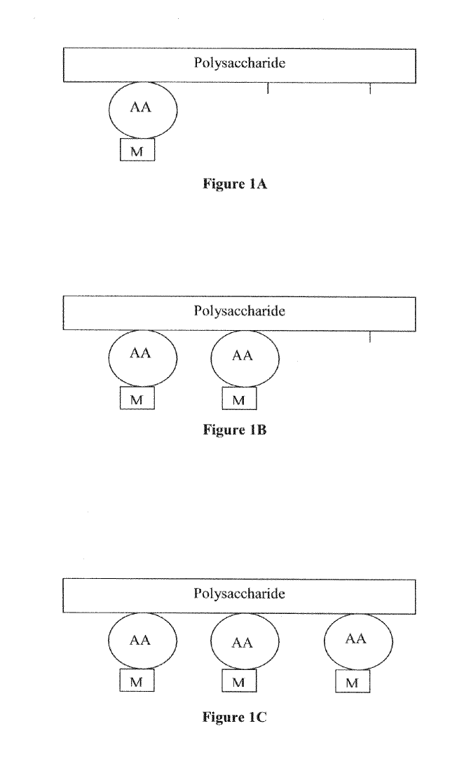

The degree of saturation of amino acid attachment points on the polysaccharide may vary. For example, as shown in FIG. 1A, only one amino acid may be attached. Very low degrees of saturation, such as 5% or less, 10% or less, or 20% or less may also be achieved. FIG. 1B illustrates a conjugate with an intermediate degree of saturation, such as approximately 30%, approximately 40%, approximately 50%, or approximately 70%. FIG. 1C illustrates a conjugate with very high degrees of saturation, such as 80% or greater, 90% or greater, 95% or greater, or substantially complete saturation. Although in FIG. 1 each amino acid has a conjugated metal, in many actual examples, there will be degrees of saturation of the available amino acids by the metal, such as less than 5%, 10% or 20%, approximately 30%, approximately 40%, approximately 50%, or approximately 70%, greater than 80%, 90%, 95%, or substantially complete saturation.

The metal may be any metal atom or ion or compound containing a metal that can be conjugated by the O-groups of the amino acid monomers. In specific embodiments, the metal may be a transition metal, such as platinum, iron, gadolinium, rhenium, manganese, cobalt, indium, gallium, or rhodium. The metal may be a therapeutic metal. It may be part of a larger molecule, such as a drug. The metal may be conjugated to the polysaccharide-amino acid backbone via O-groups of the amino acid monomers.

In one embodiment, the metal may be between 15 per cent to about 30 per cent, by weight of the polysaccharide conjugate.

In one example embodiment, the conjugate includes chondroitin A covalently bonded to aspartic acid monomers, which conjugate platinum in a platinum-containing compound. In one variation the platinum may be platinum (II) and in another variation the platinum may be platinum (IV).

The conjugate may be water soluble. For example, it may have a solubility of at least approximately 20 mg (metal equivalent)/ml water. The conjugate may be provided in a variety of forms, such as an aqueous solution or a powder. The conjugate and its formulations may be sterilized. For example, it may be provided as a sterilized powder.

The conjugate may be synthesized, according to one embodiment of the invention, by separately covalently bonding one or more amino acid monomers to a polysaccharide. Then a metal may be provided for conjugation by the amino acid monomers. According to another embodiment of the invention, the metal may be conjugated to the amino acid monomers, then one or more of the amino acid monomers may be covalently bonded to the polysaccharide.

Conjugates of the present invention may he used to kill cancer or tumor cells and thus may treat cancer or tumors. Conjugates may target tumors, particularly solid tumors. This may be verified, for example, through radiolabeled variations of the compounds, such as a polysaccharide-amino acid backbone conjugated to ^{99m}Tc, which allows gamma imaging. Cytotoxic agents with a metal component may be conjugated to the polysaccharide-amino acid backbone to reduce their cytotoxic effects. For example, the cytotoxic agents maybe released gradually from the polysaccharide, decreasing acute systemic toxicity. Additionally, the therapeutic index (toxicity/efficacy) of drugs with poor water solubility or tumor targeting capacity may be increased by conjugating those drugs to the polysaccharide-polymer backbone.

In specific example embodiments, platinum-containing conjugates may be able to inhibit cancer cell growth at lower doses than cisplatin. Further, platinum-containing conjugates may also be able to inhibit cell growth of cisplatin-resistant cancer cells, particularly ovarian cancer cells.

Any type of cancer or tumor cell may be killed or have its growth inhibited by selected conjugates of the present invention. However, solid tumors may respond best to these conjugates. Example cancers that may be susceptible to certain conjugates of the present invention include: ovarian cancer, cisplatin-resistant ovarian cancer, pancreatic cancer, breast cancer, sarcoma, uterine cancer, and lymphoma.

In addition to cancer, certain conjugates of the present invention may be able to target and inhibit cells involved in the development and progression of the following diseases: HIV, autoimmune diseases (e.g. encephalomyelitis, vitiligo, scleroderma, thyroiditis, and perforating collagenosis), genetic diseases (e.g xeroderma pigmentosum and glucose-6-phosphate dehydrogenase deficiency), metabolic diseases (e.g. diabetes mellitus), cardiovascular diseases, neuro/psychiatric diseases and other medical conditions (e.g. hypoglycemia and hepatic cirrhosis).

### EXAMPLES

The following examples are included to demonstrate specific embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Synthesis of Platinum Analogue (II) and (IV)-polysaccharide Conjugate Method A

Cis-1,2-Diaminocyclohexane sulfatoplatinum (II) (cis-1,2-DACH-Pt.SO₄) was synthesized via a two-step procedure. In the first step, cis-1,2-DACH-PtI₂ complex was synthesized by mixing a filtered solution of K₂PtCl₄ (5.00 g, 12 mmol) in 120 ml of deionized water with KI (20.00 g in 12 ml of water, 120 mol) and was allowed to stir for 5 min. To this solution one equivalent of the cis-1,2-DACH (1.37 g, 1.487 ml, 12 mmol) was added. The reaction mixture was stirred for 30 min at room temperature. The obtained yellow solid was separated by filtration and then washed with a small amount of deionized water. The final product was dried under vacuum, which yielded cis-1,2-DACH-PtI₂ (6.48 g, 96%). In the second step, cis-1,2-DACH-PtI₂ (without further purification from step 1, 6.48 g, 11.5 mmol) was added as a solid to an aqueous solution of Ag₂SO₄ (3.45 g, 11 mmol). The reaction mixture was left stirring overnight at room temperature. The AgI was removed by filtration and the filtrate was freeze dried under vacuum, which yielded yellow cis-1,2-DACH-Pt(II)SO₄ (4.83 g, 99%). Elemental analysis showed Pt: 44.6% (w/w).

To a stirred solution of chondroitin (1 g, MW. 30,000-35,000) in water (5 ml), sulfo-NHS (241.6 mg, 1.12 mmol) and 3-ethylcarbodiimide 1-ethyl-3-(3-dimethylaminopropyl)carbodiimide-HCl (EDC) (218.8 mg, 1.15 mmol) (Pierce Chemical Company, Rockford, Ill.) were added. L-aspartic acid sodium salt (356.8 mg, 1.65 mmol) was then added. The mixture was stirred at room temperature for 24 hours. The mixture was dialyzed for 48 hours using a Spectra/POR molecular porous membrane with cut-off at 10,000 (Spectrum Medical Industries Inc., Houston, Tex.). After dialysis, the product was filtered and freeze dried using lyophilizer (Labconco, Kansas City, Mo.). The product, aspartate-chondroitin (polysaccharide), in the salt form, weighed 1.29 g. A similar technique was used to prepare condroitin having glutamic acid and alanine, glutamic acid and asparagine, glutamic acid and glutamine, glutamic acid and glycine, and glutamic acid and one aspartic acid conjugated with alanine, asparagine, glutamine, and glycine.

Cis-1,2-DACH-Pt (II) SO₄ (500 mg, 1.18 mmol) was dissolved in 10 ml of deionized water, and a solution of aspartate-chondroitin (1.00 g in 15 ml of deionized water) was added. The solution was left stirring for 24 hr at room temperature. After dialysis (MW: 10,000) and lyophilization, the yield of cis-1,2-DACH-Pt (II)-polysaccharide was 1.1462 g.

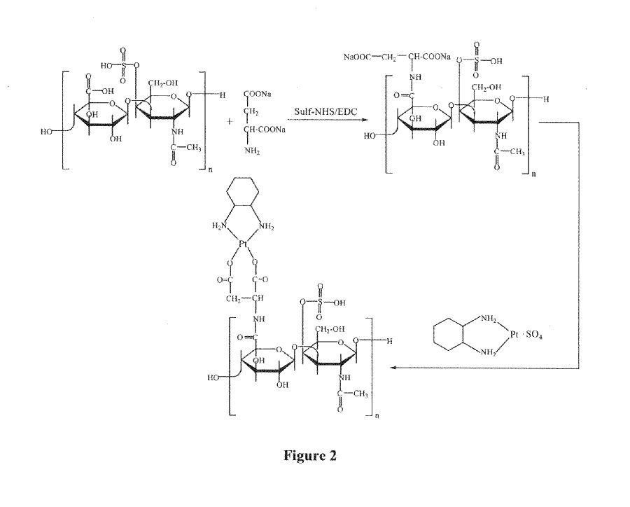

The platinum-polysaccharide conjugate, Cis-1,2-DACH-dichloro-Pt (IV)-aspartate-chondroitin (PC) was synthesized as follows: the above solution was added dropwise 2.5 ml of 30% aqueous hydrogen peroxide. After 24 hr, HCl (75 ml of 0.02 N) was added and left stirring for 24 hr at room temperature, dialyzed (MW: 10,000) by deionized water for overnight and freeze dried under vacuum. The final product obtained was 1.15 g. Elemental analysis showed Pt: 21.87% (w/w). The synthetic scheme is shown in FIG. 2.

### Method B.

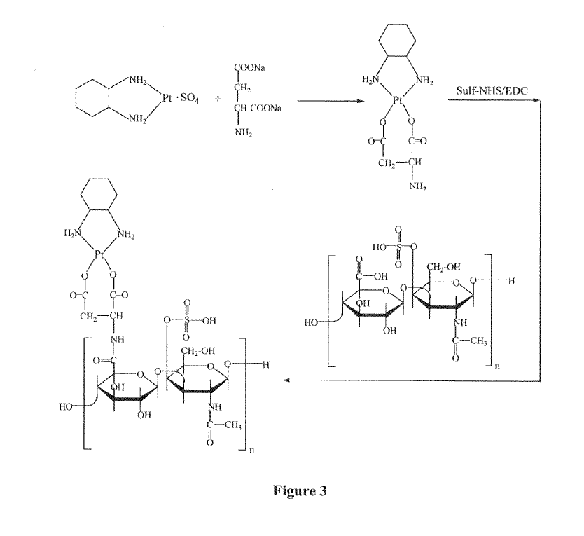

The Cis-1,2-DACH-Pt (II) SO₄ or Cis-1,2-DACH-dichloro-Pt (IV) (500 mg, 1.18 mmol) was dissolved in 10 ml of deionized water, and a solution of aspartic acid (67 mg, 0.5 mmol) in 2 ml of deionized water was added. The solution was left stirring for 24 hr at room temperature. After dialysis and lyophilization, the cis-1,2-DACH-Pt-aspartate was reacted with chondroitin (1 g, MW. 30,000-35,000) in water (5 ml), sulfo-NHS (241.6 mg, 1.12 mmol) and 3-ethylcarbodiimide 1-ethyl-3-(3-dimethylaminopropyl) carbodiimide-HCl (EDC) (218.8 mg, 1.15 mmol) (Pierce Chemical Company, Rockford, Ill.). The synthetic scheme is shown in FIG. 3.

### Example 2

### In Vitro Cell Culture Assay

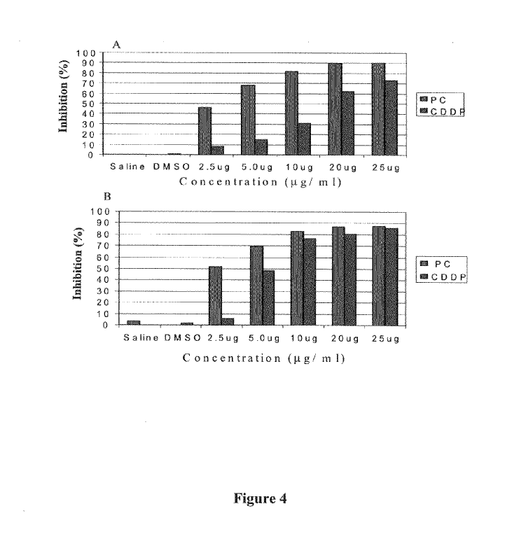

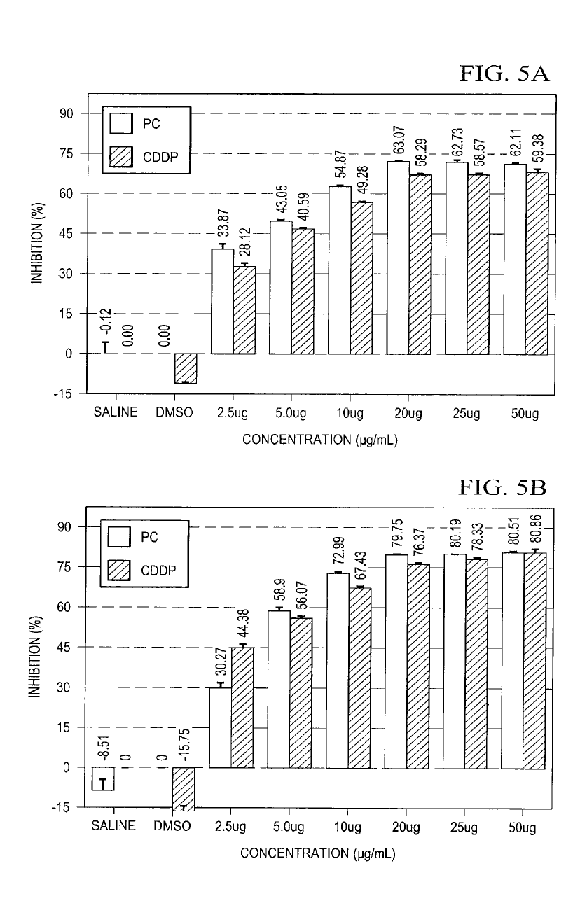

To evaluate cytotoxicity of cisplatin and platinum (II)-polysaccharide conjugate (PC) prepared as described above using Method A against mammary tumor cells, two human tumor cell lines were selected: the 2008 line and its platinum-resistant subline, 2008-c13. All cells were cultured at 37° C. in a humidified atmosphere containing 5% CO₂ in RPMI 1640 medium supplemented with 10% fetal bovine serum and glutamine (2 mM). 2008 or 2008.C13 cells were seeded into 96-well plates (4,000 cells/well) and maintained in RPMI 1640 medium for 24 hours. Next, cells were treated with PC or CDDP at concentrations of 2.5, 5, 10, 20, 25, and 50 μg/mL for 48 and 72 hours. Controls were treated with DMSO or PBS. After cells were treated, their growth and viability were determined by incubating the cells for 1 to 2 hours at 37° C. with 20 μL of tetrazolium substrate. Absorbance was measured at 450 nm using a 96-well Synergy HT-microplate reader (Biotek, Winooski, Vt.). The rate of cell growth inhibition was expressed as a percentage as follows: %=100−(OD_{controls}−OD_{treated cells})/OD_{controls}. The experiments were repeated separately three times. Methylene tetrazolium (MTT) dye assay determined the amount of viable cells. Cellular protein content was determined by Lowry assay. The drug concentration that inhibits 50% of cell growth (IC-50) was then determined. Data are expressed as the percentage differences compared with controls (OD of cells after treatment/OD of cells without treatment). An illustrated cell inhibition curves are shown in FIGS. 4, 5A and 5B.

The findings showed that the sensitivity of cells to exposure to the IC-50 of platinum-polysaccharide conjugate (PC) was 5.7 times greater than that to the exposure to the IC-50 of cisplatin (CDDP) (FIG. 4) in the platinum-resistant ovarian cancer cell line but not in the platinum-sensitive ovarian cancer line (2008) (FIG. 5A and FIG. 5B). In particular, concentrations of platinum-polysaccharide conjugate (PC) at 2.5 and 5 μg/mL enhanced tumor killing by 5.9 and 4.6 times, respectively, at 48 hours compared with cisplatin (CDDP) (FIG. 4A) and by 9.3 and 1.5 times, respectively, at 72 hours compared with cisplatin (CDDP) (FIG. 4B). The data indicated that low doses of platinum-polysaccharide conjugate (PC) significantly inhibit cell growth of platinum-resistant ovarian cancer cells.

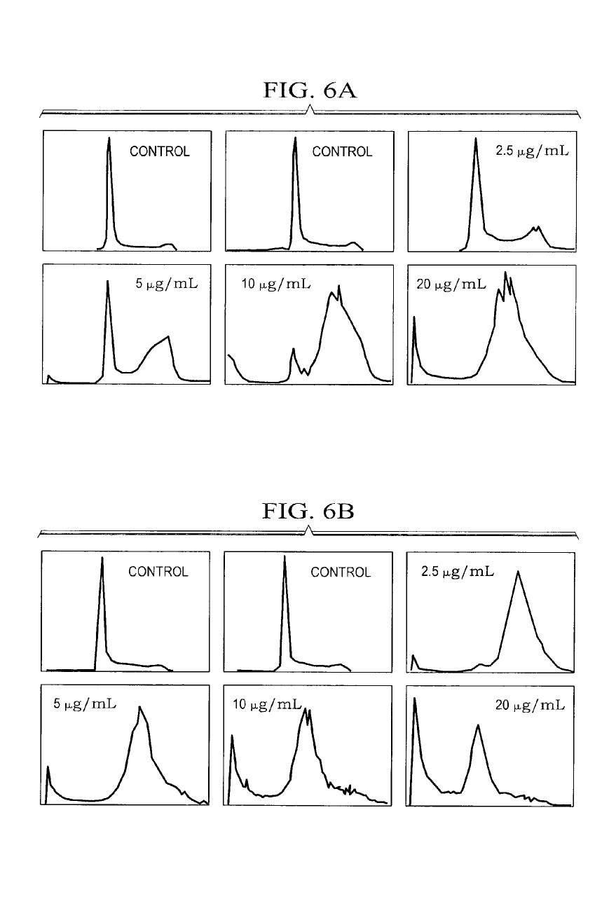

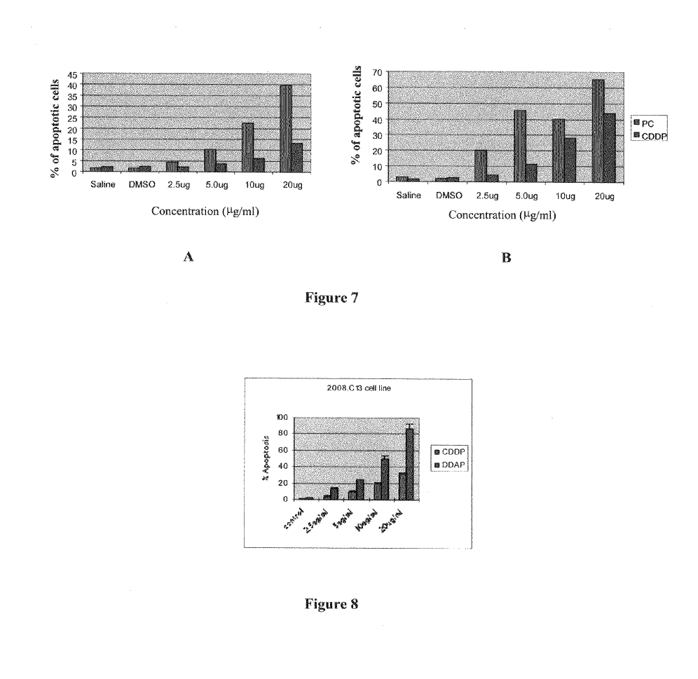

To determine the effectiveness of platinum-polysaccharide conjugate (PC) against platinum-resistant ovarian cancer cells, 2008-c13 cells (0.5×10⁻⁶) were treated with platinum-polysaccharide conjugate (PC). The cells were trypsinized and centrifuged at 2500 rpm for 5 min. After being washed with 1×PBS two times, cells were fixed with 70% ethanol overnight, washed twice with 1×PBS, and resuspended in 1 mL of propidium iodide (PI) solution (1×10⁶ cells/mL). RNase (20 μg/mL) solution was added followed by 1 mL of propydium iodide solution (PI, 50 μg/mL in PBS). Samples were incubated at 37° C. for 15 min, and PI fluorescence was analyzed using a EPIS XL analyzer. Compared to cisplatin (CDDP), platinum (IV)-polysaccharide conjugate at low concentrations (2.5 and 5 μg/mL) significantly enhanced the apoptotic effect on platinum-resistant ovarian cancer cells (FIGS. 6A, 6B, and 7).

These results were confirmed by TUNEL assay, which, after 48 hours of treatment, shows a clear dose-dependent increase of apoptotic cells was detected after exposure to both drugs. However, when compared at each dose, platinum-polysaccharide conjugate (PC) treated group had many more cells experiencing apoptosis (P<0.05) (FIG. 8).

### Example 3

### Evaluation of Anticancer Effect Using Breast Tumor-Bearing Rat Model

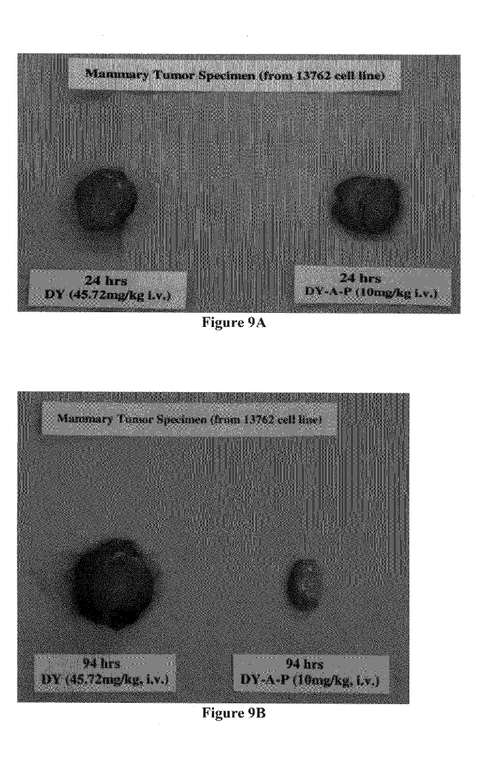

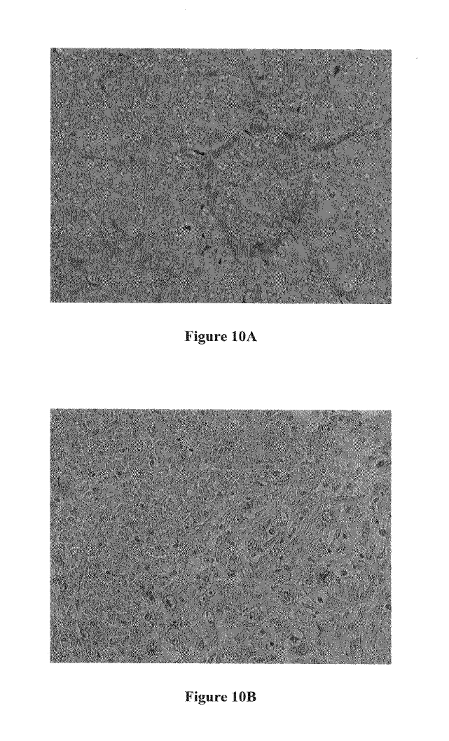

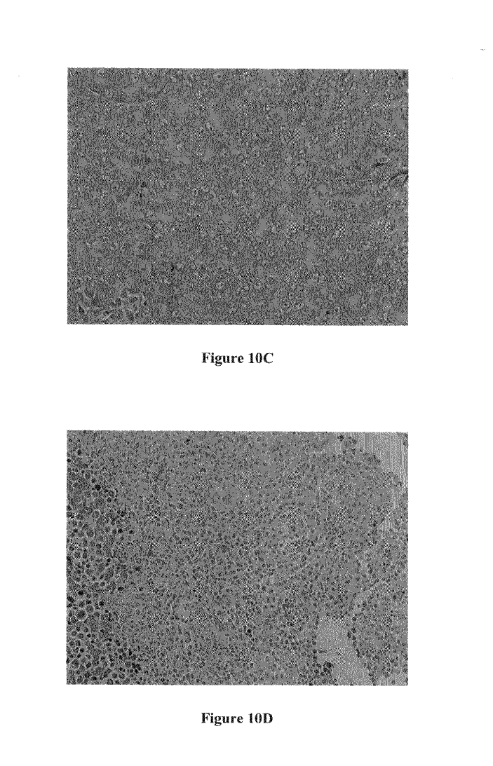

Female Fischer 344 rats (125-175 g) were inoculated with breast cancer cells (13762NF, 10⁶ cells/rat, s.c. in the hind leg). After 15-20 days and a tumor volume of 1 cm, the breast tumor-bearing rats were administered either the platinum-chondroitin (Platinum-polysaccharide) conjugate (PC) or chondroitin alone at doses of 10 mg Pt/kg (platinum (IV)-polysaccharide) or 45 mg/kg (chondroitin). Tumor volumes and body weight were recorded daily for sixty days. Tumor volumes were measured as [length (1)×width (w)×thickness (h)]/2. Loss of body weight of 15% is considered a chemical-induced toxic effect. The results indicate that the platinum-polysaccharide conjugate (PC) is effective in vivo against breast tumor growth (FIG. 9). After treatment with platinum-polysaccharide conjugate, tumor tissues were dissected and embedded in formalin. The tumor tissue was fixed in paraffin, and stained with hematoxylin and eosin for histological examinations. Extensive necrosis was observed at 94 hrs post-administration of platinum-polysaccharide conjugate, but not polysaccharide alone (FIG. 10).

### Example 4

### Method of Tumor Cell Death or Inhibition

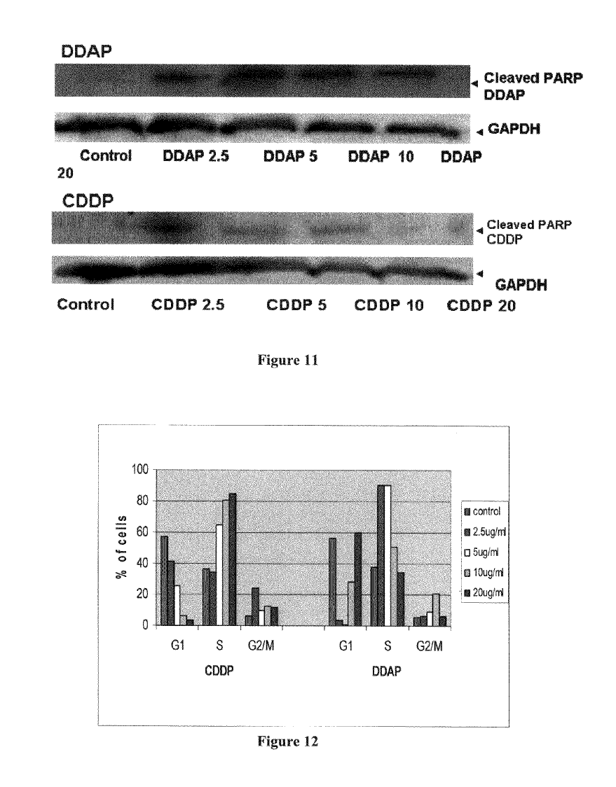

The effect of the platinum-polysaccharide conjugate of Example 1 on tumor cells was analyzed by treating 2008-c13 breast cancer cells with the conjugate, then analyzing the effects on cellular proteins through a Western blot (FIG. 11). Cleaved PARP was significantly increased in the cells treated with platinum-polysaccharide conjugate (PC), compared with cisplatin (CDDP), suggesting that platinum-polysaccharide conjugate inhibited 2008-c13 cell growth through enhancement of apoptosis in a caspase 3 dependent pathway.

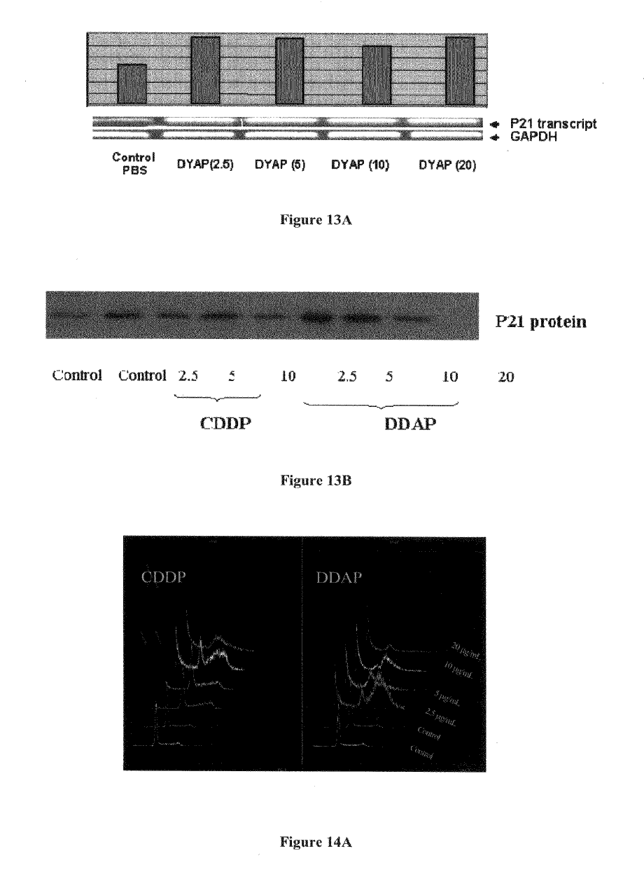

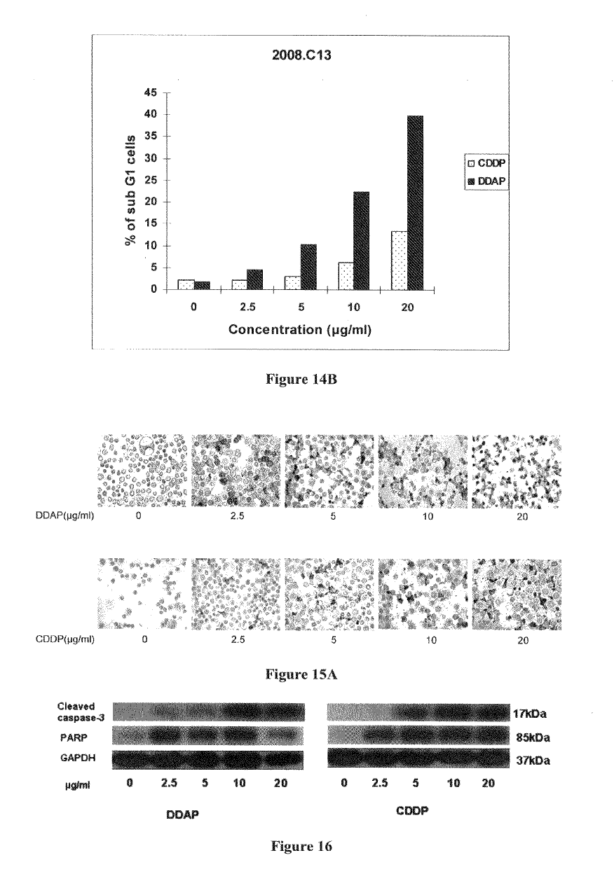

This effect was tested by flow cytometry in the 2008.C13 cell line after 48 hours and 72 hours of drug exposure. Flow cytometric analysis showed that there was a dose-dependent increase in the number of cells in the sub-G1 fraction after PC and CDDP treatments, which represents hypodiploid cells and indicates the induction of apoptosis. However, the use of PC, compared with CDDP, resulted in a more pronounced increase in the sub-G1 fraction at the same doses (FIG. 14).

DNA fragmentation typical of apoptosis was further determined by the TUNEL assay in three independent experiments. A clear dose-dependent increase in the number of apoptotic cells was detected after exposure to both drugs. However, when compared at each dose, the PC-treated cells exhibited much higher levels of apoptosis (P<0.05) (FIG. 15).

To determine whether apoptosis is induced through a caspase-3-dependent pathway followed by the cleavage of PARP, levels of cleaved caspase-3 and PARP, which form after caspase-3 activation, were determined by Western blot analysis. PARP is a 113-kDa nuclear protein that has been shown to be specifically cleaved to an 85-kDa fragment during caspase-3-dependent apoptosis. After cells were exposed to CDDP or PC for 48 hours, cleaved PARP was present at each dose. In the CDDP-treated group, cleaved PARP expression increased from 2.5 μg/mL to 20 μg/mL and cleaved caspase-3 was expressed in a pattern similar to that of PARP. In the PC-treated group, the expression of cleaved caspase-3 was comparable to that in the CDDP-treated group, except for the lower expression seen at 5 μg/mL of PC. Although cleaved PARP expression induced by high-dose (20 μg/mL) PC appeared to be lower than that induced by low-dose PC, no such difference was detected in its upstream cleaved caspase-3 expression (FIG. 16).

In a further test on 2008-c13 breast cancer cells in vitro, flow cytometric analysis showed that cells significantly arrested in S-phase after exposure to platinum-polysaccharide conjugate (PC) at 48 hours (FIG. 12). The highest levels of S-phase blockage happened at lower dosages of 2.5 and 5 μg/ml (90.3% and 90.1%). When compared with cisplatin (CDDP), the effect of platinum-polysaccharide conjugate (PC) on arresting cells in S-phase is significantly different.

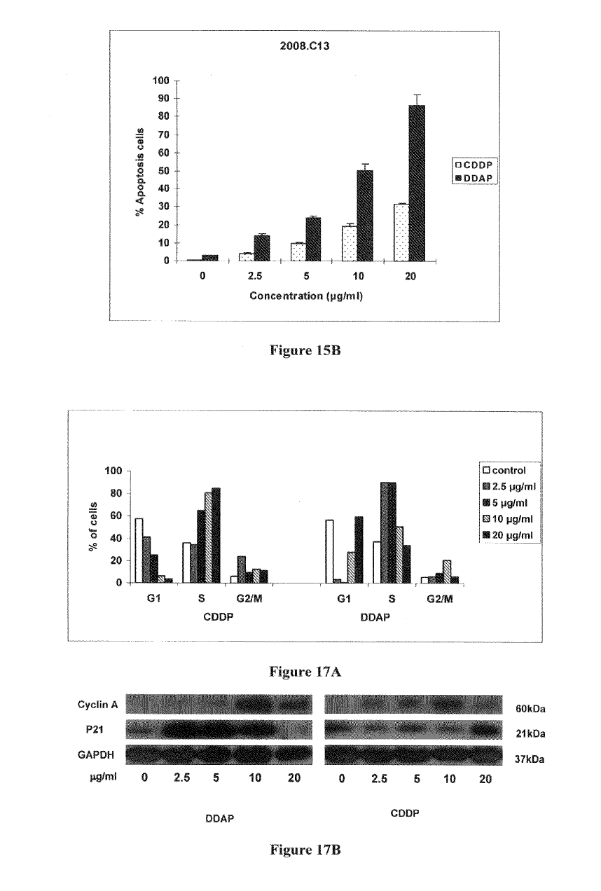

Specifically, DNA content was analyzed by flow cytometry 48 hours after 2008.C13 cells were treated with PC or CDDP. Exposure to CDDP induced cell arrest in the S-phase and increased the sub-G1 fraction at the 5 μg/mL dose, but not at the lowest dose, 2.5 μg/mL. The numbers of cells arrested in the S phase and sub-G1 fraction increased continuously as the CDDP dose increased, with the maximal S-phase arrest (84.8%) occurring at 20 μg/mL. After cells were exposed to PC for 48 hours, the highest levels of S-phase block occurred at the lower doses (2.5 μg/mL [90.3%] and 5 μg/mL [90.1%]). At higher doses (10 and 20 μg/mL), the level of S-phase arrest steadily decreased as the sub-G1 fraction increased. This can be explained by the fact that under the strong stress of high-dose PC, cells underwent apoptosis promptly and directly before they were arrested in the S-phase (FIG. 17).**

To elucidate the mechanism underlying S-phase arrest caused by CDDP and PC in 2008.C13 cells, the expression of p21 and cyclin A, which are important for cell-cycle regulation in the S phase, was examined in 2008.C13 cells after 48 hours of drug exposure. Neither p21 nor cyclin A expression was related to the extent of S-phase arrest after CDDP treatment. After PC treatment, however, p21 and cyclin A expression were directly related to the extent of S-phase arrest: p21 was up-regulated with maximal S-phase arrest after low-dose PC treatment, but not after high doses; cyclin A was up-regulated after high-dose PC treatment and was maintained at a low level after low-dose PC treatment (FIG. 17B 2008-c13 breast cancer cells treated with platinum-polysaccharide conjugate (PC) showed increased p21 expression at both transcriptional (FIG. 13A) and protein expression levels (FIG. 13B) as compared to cells treated with cisplatin (CDDP).

Although only exemplary embodiments of the invention are specifically described above, it will be appreciated that modifications and variations of these examples are possible without departing from the spirit and intended scope of the invention.

What is claimed is:

1. A method of killing a cancer cell comprising administering to the cell an effective amount of a polysaccharide conjugate comprising:
a polysaccharide;
at least one monomeric amino acid having an O-group covalently bound to the polysaccharide; and
at least one metal conjugated by the O-group of the amino acid.

2. The method of claim 1, wherein the cancer cell is a cisplatin-resistance cancer cell.

3. The method of claim 1, wherein the cancer cell is located in a human patient and administering comprises providing the polysaccharide conjugate to the human patient.
